(19) United States
(12) Patent Application Publication
(10) Pub. No.: US 2002/0157137 A1
(43) Pub. Date: Oct. 24, 2002

(54) Nucleic acids that control reproductive development in plants

(76) Inventors: Yong-Hwan Moon, Albany, CA (US); Lingjing Chen, Albany, CA (US); Zinmay Renee Sung, Hillsborough, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/828,068

(22) Filed: Apr. 6, 2001

(51) Int. Cl. C07H 21/04; C12N 15/82; C12N 15/87; C12N 15/09; C12N 15/63; C12N 15/29

(52) U.S. Cl. 800/290; 536/23.6; 435/320.1; 435/468; 800/298; 800/278; 800/286

(57) ABSTRACT

The invention provides novel genes uselful in and methods of modulating reproductive development in plants.

This invention was made with United States government support under Grant No. 99-35301-7984 awarded by the United States Department of Agriculture. The United States government has certain rights to this invention.

### FIELD OF THE INVENTION

The present invention is directed to plant genetic engineering. In particular, it relates to novel genes controlling reproductive development in rice and modulation of expression of genes controlling reproductive development in rice.

### BACKGROUND OF THE INVENTION

The transition from rosette to early inflorescence is considered to be the vegetative-to-reproductive transition. It is regulated by many flowering-time genes, that is, floral repression and floral promotion genes (or early- and late-flowering genes, respectively) (Koornneef et al., Mol. Gen. Genet. 229:57-66 (1991); Zagotta, et al., Aust. J. Plant Physiol. 19:411-418 (1992)). Loss-of-function mutations in floral repression genes, such as EARLY FLOWER 1 (ELF1), cause early flowering, whereas mutations in floral promotion genes, such as CONSTANS (CO), delay transition from the rosette-to-inflorescence stage. In addition, two EMBRYONIC FLOWER (EMF) genes, EMF1 and EMF2, are proposed to be involved in this process as floral repressors, suppressing the onset of reproductive development (Sung et al., Science 258:1645-1647 (1992); Martinez-Zapater et al. In Arabidopsis, E. M. Meyerowitz and C. R. Somerville, eds (Cold Spring Harbor, N.Y.: Cold Spring Harbor Laboratory Press), pp 403-433 (1994); Castle, et al., Flowering Newslet. 19:12-19 (1995); Yang, et al., Dev. Biol. 169:421-435 (1995)). Based on this floral repressor concept, vegetatively growing plants must decrease EMF1 and EMF2 activities to initiate reproductive growth. It has been proposed that the floral repression genes maintain, whereas floral promotion genes inhibit, EMF1 and EMF2 activities. A balance of these gene actions would cause a gradual decline in EMF activities and determine the time of vegetative-to-reproductive transition.

The transition from inflorescence to flower is regulated by flower meristem identity genes, such as LEAFY (LFY), APETALA1 (AP1), AP2, and CAULIFLOWER (CAL) (Irish, et al., Plant Cell 2:741-753 (1990); Mandel, et al., Nature 360:273-277 (1992); Bowman, et al., Development 119:721-743 (1993); Jofuku, et al., Plant Cell 6:1211-1225 (1994)). Mutants with defective LFY, AP1, AP2, or AP1 CAL genes are impaired in flower initiation; thus, inflorescence-like or flowerlike shoots, instead of flowers, initiate peripherally from the apical meristem during the late-inflorescence phase. In addition to these genes, the TERMINAL FLOWER1 (TFL1) gene is reported to negatively regulate meristem identity gene function in inflorescence development. Both the primary shoot and the lateral shoots in tfl1 mutants terminate in a flower, reflecting a precocious inflorescence-to-flower transition (Alvarez et al., Plant J. 2:103-116 (1992)). Molecular data have shown that the LFY gene is ectopically expressed in the entire apical meristem of tfl1 primary and lateral shoots, which is consistent with the tfl1 phenotype (Bradley, et al., Science 275:80-83 (1997)). Thus, TFL1 functions to maintain inflorescence development. Mutants impaired in EMF1 or EMF2 produce a reduced inflorescence and a terminal flower, indicating a role for the EMF genes in delaying the inflorescence-to-flower transition.

In light of the above, it is clear that EMF genes play an important role in reproductive development in plants. Control of the expression of the genes is therefore useful in controlling flowering and other functions in plants. The present invention features an OsEMF1 gene isolated from rice and methods for controlling flowering and reproduction using the same. These and other advantages are provided by the present application.

### SUMMARY OF THE INVENTION

The present invention provides methods of modulating reproductive development such as shoot architecture, flowering time, seed yields and other traits in plants. The methods involve providing a plant comprising a recombinant expression cassette containing an OsEMF1 nucleic acid linked to a plant promoter.

In some embodiments, expression of the OsEMF1 nucleic acids of the invention are used to enhance expression of an endogenous OsEMF1 gene or gene product activity. In these embodiments, the nucleic acids are used to inhibit or delay transition to a reproductive state and can be used to promote vegetative growth of the plant. Alternatively, transcription of the OsEMF1 nucleic acid inhibits expression of an endogenous OsEMF1 gene or the activity of the encoded protein. These embodiments are particularly useful in promoting the transition to a reproductive state and, for instance, promoting uniform flowering, obtaining early flowering plants, and generating plant varieties with more branches.

In the expression cassettes, the plant promoter may be a constitutive promoter, for example, the CaMV 35S promoter. Alternatively, the promoter may be a tissue-specific or an inducible promoter. For instance, the promoter sequence from the OsEMF1 genes disclosed here can be used to direct expression in relevant plant tissues.

The invention also provides seed or fruit produced by the methods described above. The seed or fruit of the invention comprise a recombinant expression cassette containing an OsEMF1 nucleic acid.

Definitions

The phrase “nucleic acid sequence” refers to a single or double-stranded polymer of deoxyribonucleotide or ribonucleotide bases read from the 5′ to the 3′ end. It includes chromosomal DNA, self-replicating plasmids, infectious polymers of DNA or RNA and DNA or RNA that performs a primarily structural role..

A “promoter” is defined as an array of nucleic acid control sequences that direct transcription of an operably linked nucleic acid. As used herein, a “plant promoter” is a promoter that functions in plants. Promoters include necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “plant” includes whole plants, plant organs (e.g., leaves, stems, flowers, roots, etc.), seeds and plant cells and progeny of same. The class of plants which can be used in the method of the invention is generally as broad as the class of higher plants amenable to transformation techniques, including angiosperms (monocotyledonous and dicotyledonous plants), as well as gymnosperms. It includes plants of a variety of ploidy levels, including polyploid, diploid, haploid and hemizygous.

A polynucleotide sequence is “heterologous to” an organism or a second polynucleotide sequence if it originates from a foreign species, or, if from the same species, is modified from its original form. For example, a promoter operably linked to a heterologous coding sequence refers to a coding sequence from a species different from that from which the promoter was derived, or, if from the same species, a coding sequence which is different from any naturally occurring allelic variants.

A polynucleotide “exogenous to” an individual plant is a polynucleotide which is introduced into the plant by any means other than by a sexual cross. Examples of means by which this can be accomplished are described below, and include Agrobacterium-mediated transformation, biolistic methods, electroporation, and the like. Such a plant containing the exogenous nucleic acid is referred to here as an R₁ generation transgenic plant. Transgenic plants which arise from sexual cross or by selfing are descendants of such a plant.

A “OsEMF1 nucleic acid” or “OsEMF1 polynucleotide sequence” of the invention is a subsequence or full length polynucleotide sequence of a gene which encodes a polypeptide involved in control of reproductive development and which, when mutated, promotes a transition to a reproductive state, e.g., flowering, in plants. An exemplary nucleic acid of the invention is the Oryzae EMF1 sequence disclosed below. OsEMF1 polynucleotides of the invention are defined by their ability to hybridize under defined conditions to the exemplified nucleic acids or PCR products derived from them. An OsEMF1 polynucleotide is typically at least about 30-40 nucleotides to about 4500 nucleotides, usually about 3800 to 4000 nucleotides in length. The nucleic acids contain coding sequence of from about 100 to about 28000 nucleotides, often from about 500 to about 1000 nucleotides in length.

In the case of both expression of transgenes and inhibition of endogenous genes (e.g., by antisense, or sense suppression) one of skill will recognize that the inserted polynucleotide sequence need not be identical, but may be only “substantially identical” to a sequence of the gene from which it was derived. As explained below, these substantially identical variants are specifically covered by the term OsEMF1 nucleic acid.

In the case where the inserted polynucleotide sequence is transcribed and translated to produce a functional polypeptide, one of skill will recognize that because of codon degeneracy a number of polynucleotide sequences will encode the same polypeptide. These variants are specifically covered by the terms “OsEMF1 nucleic acid”. In addition, the term specifically includes those sequences substantially identical (determined as described below) with an OsEMF1 polynucleotide sequence disclosed here and that encode polypeptides that are either mutants of wild type OsEMF1 polypeptides or retain the function of the OsEMF1 polypeptide (e.g. resulting from conservative substitutions of amino acids in the OsEMF1 polypeptide). In addition, variants can be those that encode dominant negative mutants as described below.

Two nucleic acid sequences or polypeptides are said to be “identical” if the sequence of nucleotides or amino acid residues, respectively, in the two sequences is the same when aligned for maximum correspondence as described below. The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence over a comparison window, as measured using one of the following sequence comparison algorithms or by manual alignment and visual inspection. When percentage of sequence identity is used in reference to proteins or peptides, it is recognized that residue positions that are not identical often differ by conservative amino acid substitutions, where amino acids residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated according to, e.g., the algorithm of Meyers & Miller, Computer Applic. Biol. Sci. 4:11-17 (1988) e.g., as implemented in the program PC/GENE (Intelligenetics, Mountain View, Calif., USA)..

The phrase “substantially identical,” in the context of two nucleic acids or polypeptides, refers to sequences or subsequences that have at least 60%, preferably 80%, most preferably 90-95% nucleotide or amino acid residue identity when aligned for maximum correspondence over a comparison window as measured using one of the following sequence comparison algorithms or by manual alignment and visual inspection. This definition also refers to the complement of a test sequence, which has substantial sequence or subsequence complementarity when the test sequence has substantial identity to a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection.

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle, J. Mol. Evol. 35:351-360 (1987). The method used is similar to the method described by Higgins & Sharp, CABIOS 5:151-153 (1989). The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art.

The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

(see, e.g., Creighton, Proteins (1984)).

An indication that two nucleic acid sequences or polypeptides are substantially identical is that the polypeptide encoded by the first nucleic acid is immunologically cross reactive with the antibodies raised against the polypeptide encoded by the second nucleic acid. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below.

The phrase “selectively (or specifically) hybridizes to” refers to the binding, duplexing, or hybridizing of a molecule only to a particular nucleotide sequence under stringent hybridization conditions when that sequence is present in a complex mixture (e.g., total cellular or library DNA or RNA).

The phrase “stringent hybridization conditions” refers to conditions under which a probe will hybridize to its target subsequence, typically in a complex mixture of nucleic acid, but to no other sequences. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Probes, “Overview of principles of hybridization and the strategy of nucleic acid assays” (1993). Generally, highly stringent conditions are selected to be about 5-10° C. lower than the thermal melting point (Tₘ) for the specific sequence at a defined ionic strength pH. Low stringency conditions are generally selected to be about 15-30° C. below the Tₘ. The Tₘ is the temperature (under defined ionic strength, pH, and nucleic concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. For selective or specific hybridization, a positive signal is at least two times background, preferably 10 time background hybridization.

Nucleic acids that do not hybridize to each other under stringent conditions are still substantially identical if the polypeptides which they encode are substantially identical. This occurs, for example, when a copy of a nucleic acid is created using the maximum codon degeneracy permitted by the genetic code. In such cased, the nucleic acids typically hybridize under moderately stringent hybridization conditions.

In the present invention, genomic DNA or cDNA comprising OsEMF1 nucleic acids of the invention can be identified in standard Southern blots under stringent conditions using the nucleic acid sequences disclosed here. For the purposes of this disclosure, suitable stringent conditions for such hybridizations are those which include a hybridization in a buffer of 40% formamide, 1 M NaCl, 1% SDS at 37° C., and at least one wash in 0.2×SSC at a temperature of at least about 50° C., usually about 55° C. to about 60° C., for 20 minutes, or equivalent conditions. A positive hybridization is at least twice background. Those of ordinary skill will readily recognize that alternative hybridization and wash conditions can be utilized to provide conditions of similar stringency.

A further indication that two polynucleotides are substantially identical is if the reference sequence, amplified by a pair of oligonucleotide primers, can then be used as a probe under stringent hybridization conditions to isolate the test sequence from a cDNA or genomic library, or to identify the test sequence in, e.g., a northern or Southern blot.

### BRIEF DESCRIPTION OF THE DRAWINGS

FIG. 1 describes the OsEMF1 nucleotide coding seqeunce and the peptide amino acid sequence.

### DESCRIPTION OF THE SPECIFIC EMBODIMENTS

This invention provides molecular strategies for controlling reproductive development, in particular flowering time, shoot development and seed yield, in plants, particularly monocotolydenous plants and preferably rice. The invention has wide application in agriculture. For example, enhanced expression of genes of the invention is useful to alter flowering time, shoot development and seed yield in plants, particularly monocotolydenous plants and preferably rice. Controlling or inhibiting expression of the genes is useful to generate new varieties of rice having differing flowering times and seed yield.

The present invention is based, at least in part, on the discovery of mutations, embryonic flowering (emf), and the subsequent cloning of the genes involved. A genetic model for the control of vegetative-to-reproductive transition has been proposed (Martinez-Zapater et al., In Arabidopsis, E. M. Meyerowitz and C. R. Somerville, eds (Cold Spring Harbor, N.Y.: Cold Spring Harbor Laboratory Press), pp. 403-433 (1994)). The main scheme of the model is that flowering is a default state and is negatively regulated by floral repressors. The hypothesis assumes that vegetative development is maintained as a result of the suppression of reproductive development. The OsEMF gene products are floral repressors because weak emf mutants produce an inflorescence directly after germination. For example, severe emf1 alleles cause the shoot to shift further into the reproductive state than do weak alleles, as evidenced by several distinct floral characteristics, including lack of stipules and trichomes on lateral organs, carpelloidy of lateral organs, direct development of a single flower or pistil, and precocious expression of floral genes (Chen et al. The Plant Cell 9:2011-2024 (1997)).

To flower, juvenile plants must acquire floral competence first (McDaniel, et al., Dev. Biol. 153:59-69 (1992)). Without wishing to be bound by theory it is proposed that the products of OsEMF1 genes, specify the level of floral competence, which must be abated to a level to enable the partial derepression of floral target genes for LFY to initiate flower development. In the absence of LFY, as in lfy and Ify ap1 plants, continued increase of floral competence would still occur, resulting in floral target gene expression and carpelloid organ formation.

Many observations indicate the existence of a gradient of “floral character” along the Arabidopsis inflorescence axis. The gradient of floral character can also be seen on the shoots of other annual plants, such as tobacco (Tran, Planta 115:87-92 (1973)). The common features seen in different plants suggest that the mechanism controlling plant shoot maturation may be a conserved one in angiosperms. This gradient effect may be interpreted as resulting from an increasing amount of floral activators or a decreasing amount of floral repressors during inflorescence development.

Without wishing to be bound by theory it is believed that the decline of floral repressor responsible for the vegetative-to-reproductive transition is also responsible for increasing the floral character during inflorescence development. For example, the differences in weak and strong emf1 phenotypes suggest that the extent of floral character corresponds with the EMF activity in Arabidopsis (Chen et al. The Plant Cell 9:2011-2024 (1997).

Epistasis of emf to floral repression and floral promotion mutations suggests that these floral genes act by modulating EMF activity to cause vegetative-to-inflorescence transition. Likewise, epistasis of emf1-2 to lfy-1, ap1-1, ap2-1, and ap1-1cal suggests that EMF1 acts downstream from those floral genes in mediating the inflorescence-to-flower transition (Chen et al.). On the other hand, EMF appears to suppress floral genes. Therefore, there seems to be a reciprocal negative interaction between EMF and floral genes in controlling the development of Arabidopsis shoots from inflorescence to flower phase. This kind of interaction is consistent with the controllers of phase switching (COPS) hypothesis, which places EMF1 in the center of the COPS activity (Schultz, et al., Development 119:745-765 (1993)).

The COPS hypothesis holds that a high level of COPS activity suppresses reproductive development, allowing vegetative growth. If COPS activities continue to decline throughout the life span, the plant can progress from the rosette to inflorescence and to the flower phase. The reciprocal negative regulation between EMF and the floral genes provides a plausible mechanism for this hypothesis. During rosette growth, high EMF activity suppresses floral genes. EMF decline, mediated by the flowering-time genes, allows the activation of floral genes, which in turn suppress EMF activity, resulting in the sequential activation of other floral genes and the gradual decline of EMF activity during inflorescence and flower development.

Based on the above, it is clear that modulation of OsEMF1 activity can be used to control reproductive development in rice. Thus, isolated sequences prepared as described herein, can be used in a number of techniques, for example, to suppress or enhance endogenous OsEMF1 gene expression. Modulation of OsEMF1 gene expression or OsEMF1 activity in rice is particularly useful in controlling the transition from the vegetative to the reproductive state.

Isolation of OsEMF1 Nucleic Acids

Generally, the nomenclature and the laboratory procedures in recombinant DNA technology described below are those well known and commonly employed in the art. Standard techniques are used for cloning, DNA and RNA isolation, amplification and purification. Generally enzymatic reactions involving DNA ligase, DNA polymerase, restriction endonucleases and the like are performed according to the manufacturer's specifications. These techniques and various other techniques are generally performed according to Sambrook et al., Molecular Cloning—A Laboratory Manual, Cold Spring Harbor Laboratory, Cold Spring Harbor, N.Y., (1989).

The isolation of OsEMF1 nucleic acids may be accomplished by a number of techniques. For instance, oligonucleotide probes based on the sequences disclosed here can be used to identify the desired gene in a cDNA or genomic DNA library. To construct genomic libraries, large segments of genomic DNA are generated by random fragmentation, e.g. using restriction endonucleases, and are ligated with vector DNA to form concatemers that can be packaged into the appropriate vector. To prepare a cDNA library, mRNA is isolated from the desired organ, such as ovules, and a cDNA library which contains the OsEMF1 gene transcript is prepared from the mRNA. Alternatively, cDNA may be prepared from mRNA extracted from other tissues in which OsEMF1 genes or homologs are expressed.

The cDNA or genomic library can then be screened using a probe based upon the sequence of a cloned OsEMF1 gene disclosed here. Probes may be used to hybridize with genomic DNA or cDNA sequences to isolate homologous genes in the same or different plant species. Alternatively, antibodies raised against an OsEMF1 polypeptide can be used to screen an mRNA expression library.

Alternatively, the nucleic acids of interest can be amplified from nucleic acid samples using amplification techniques. For instance, polymerase chain reaction (PCR) technology can be used to amplify the sequences of the OsEMF1 genes directly from genomic DNA, from cDNA, from genomic libraries or cDNA libraries. PCR and other in vitro amplification methods may also be useful, for example, to clone nucleic acid sequences that code for proteins to be expressed, to make nucleic acids to use as probes for detecting the presence of the desired mRNA in samples, for nucleic acid sequencing, or for other purposes. For a general overview of PCR see PCR Protocols: A Guide to Methods and Applications. (Innis, M, Gelfand, D., Sninsky, J. and White, T., eds.), Academic Press, San Diego (1990).

Appropriate primers and probes for identifying OsEMF1 sequences from plant tissues are generated from comparisons of the sequences provided here with other related genes. Using these techniques, one of skill can identify conserved regions in the nucleic acids disclosed here to prepare the appropriate primer and probe sequences. Primers that specifically hybridize to conserved regions in OsEMF1 genes can be used to amplify sequences from widely divergent plant species.

Standard nucleic acid hybridization techniques using the conditions disclosed above can then be used to identify full length cDNA or genomic clones.

Inhibition of OsEMF1 Activity or Gene Expression

Since EMF1 genes are involved in controlling reproduction, inhibition of endogenous OsEMF1 activity or gene expression is useful in a number of contexts. For instance, inhibition of expression is useful in promoting flowering in plants.

Inhibition of OsEMF1 Gene Expression

The nucleic acid sequences disclosed here can be used to design nucleic acids useful in a number of methods to inhibit gene expression in plants. For instance, antisense technology can be conveniently used. To accomplish this, a nucleic acid segment from the desired gene is cloned and operably linked to a promoter such that the antisense strand of RNA will be transcribed. The construct is then transformed into plants and the antisense strand of RNA is produced. In plant cells, it has been suggested that antisense suppression can act at all levels of gene regulation including suppression of RNA translation (see, Bourque Plant Sci. (Limerick) 105: 125-149 (1995); Pantopoulos In Progress in Nucleic Acid Research and Molecular Biology, Vol. 48. Cohn, W. E. and K. Moldave (Ed.). Academic Press, Inc.: San Diego, Calif., USA; London, England, UK. p. 181-238; Heiser et al. Plant Sci. (Shannon) 127: 61-69 (1997)) and by preventing the accumulation of mRNA which encodes the protein of interest, (see, Baulcombe Plant Mol. Bio. 32:79-88 (1996); Prins and Goldbach Arch. Virol. 141: 2259-2276 (1996); Metzlaff et al. Cell 88: 845-854 (1997), Sheehy et al., Proc. Nat. Acad. Sci. USA, 85:8805-8809 (1988), and Hiatt et al., U.S. Pat. No. 4,801,340).

The nucleic acid segment to be introduced generally will be substantially identical to at least a portion of the endogenous OsEMF1 gene or genes to be repressed. The sequence, however, need not be perfectly identical to inhibit expression. The vectors of the present invention can be designed such that the inhibitory effect applies to other genes within a family of genes exhibiting homology or substantial homology to the target gene.

For antisense suppression, the introduced sequence also need not be full length relative to either the primary transcription product or fully processed mRNA. Generally, higher homology can be used to compensate for the use of a shorter sequence. Furthermore, the introduced sequence need not have the same intron or exon pattern, and homology of non-coding segments may be equally effective. Normally, a sequence of between about 30 or 40 nucleotides and about full length nucleotides should be used, though a sequence of at least about 100 nucleotides is preferred, a sequence of at least about 200 nucleotides is more preferred, and a sequence of about 500 to about 3500 nucleotides is especially preferred.

A number of gene regions can be targeted to suppress OsEMF1 gene expression. The targets can include, for instance, the coding regions, introns, sequences from exon/intron junctions, 5′ or 3′ untranslated regions, and the like.

Another well known method of suppression is sense cosuppression. Introduction of nucleic acid configured in the sense orientation has been recently shown to be an effective means by which to block the transcription of target genes. For an example of the use of this method to modulate expression of endogenous genes (see, Assaad et al. Plant Mol. Bio. 22: 1067-1085 (1993); Flavell Proc. Natl. Acad. Sci. USA 91: 3490-3496 (1994); Stam et al. Annals Bot. 79: 3-12 (1997); Napoli et al., The Plant Cell 2:279-289 (1990); and U.S. Pat. Nos. 5,034,323, 5,231,020, and 5,283,184).

The suppressive effect may occur where the introduced sequence contains no coding sequence per se, but only intron or untranslated sequences homologous to sequences present in the primary transcript of the endogenous sequence. The introduced sequence generally will be substantially identical to the endogenous sequence intended to be repressed. This minimal identity will typically be greater than about 65%, but a higher identity might exert a more effective repression of expression of the endogenous sequences. Substantially greater identity of more than about 80% is preferred, though about 95% to absolute identity would be most preferred. As with antisense regulation, the effect should apply to any other proteins within a similar family of genes exhibiting homology or substantial homology.

For sense suppression, the introduced sequence, needing less than absolute identity, also need not be full length, relative to either the primary transcription product or fully processed mRNA. This may be preferred to avoid concurrent production of some plants which are overexpressers. A higher identity in a shorter than full length sequence compensates for a longer, less identical sequence. Furthermore, the introduced sequence need not have the same intron or exon pattern, and identity of non-coding segments will be equally effective. Normally, a sequence of the size ranges noted above for antisense regulation is used. In addition, the same gene regions noted for antisense regulation can be targeted using cosuppression technologies.

Oligonucleotide-based triple-helix formation can also be used to disrupt OsEMF1 gene expression. Triplex DNA can inhibit DNA transcription and replication, generate site-specific mutations, cleave DNA, and induce homologous recombination (see, e.g., Havre and Glazer J. Virology 67:7324-7331 (1993); Scanlon et al. FASEB J. 9:1288-1296 (1995); Giovannangeli et al. Biochemistry 35:10539-10548 (1996); Chan and Glazer J. Mol. Medicine (Berlin) 75: 267-282 (1997)). Triple helix DNAs can be used to target the same sequences identified for antisense regulation.

Catalytic RNA molecules or ribozymes can also be used to inhibit expression of OsEMF1 genes. It is possible to design ribozymes that specifically pair with virtually any target RNA and cleave the phosphodiester backbone at a specific location, thereby functionally inactivating the target RNA. In carrying out this cleavage, the ribozyme is not itself altered, and is thus capable of recycling and cleaving other molecules, making it a true enzyme. The inclusion of ribozyme sequences within antisense RNAs confers RNA-cleaving activity upon them, thereby increasing the activity of the constructs. Thus, ribozymes can be used to target the same sequences identified for antisense regulation.

A number of classes of ribozymes have been identified. One class of ribozymes is derived from a number of small circular RNAs which are capable of self-cleavage and replication in plants. The RNAs replicate either alone (viroid RNAs) or with a helper virus (satellite RNAs). Examples include RNAs from avocado sunblotch viroid and the satellite RNAs from tobacco ringspot virus, lucerne transient streak virus, velvet tobacco mottle virus, solanum nodiflorum mottle virus and subterranean clover mottle virus. The design and use of target RNA-specific ribozymes is described in Zhao and Pick, Nature 365:448-451 (1993); Eastham and Ahlering, J. Urology 156:1186-1188 (1996); Sokol and Murray, Transgenic Res. 5:363-371 (1996); Sun et al., Mol. Biotechnology 7:241-251 (1997); and Haseloff et al., Nature, 334:585-591 (1988).

Modification of Endogenous OsEMF1 genes

Methods for introducing genetic mutations into plant genes are well known. For instance, seeds or other plant material can be treated with a mutagenic chemical substance, according to standard techniques. Such chemical substances include, but are not limited to, the following: diethyl sulfate, ethylene imine, ethyl methanesulfonate and N-nitroso-N-ethylurea. Alternatively, ionizing radiation from sources such as, X-rays or gamma rays can be used.

Alternatively, homologous recombination can be used to induce targeted gene disruptions by specifically deleting or altering the OsEMF1 gene in vivo (see, generally, Grewal and Klar, Genetics 146: 1221-1238 (1997) and Xu et al., Genes Dev. 10: 2411-2422 (1996)). Homologous recombination has been demonstrated in plants (Puchta et al., Experientia 50: 277-284 (1994), Swoboda et al., EMBO J. 13: 484-489 (1994); Offringa et al., Proc. Natl. Acad. Sci. USA 90: 7346-7350 (1993); and Kempin et al. Nature 389:802-803 (1997)).

In applying homologous recombination technology to the genes of the invention, mutations in selected portions of an OsEMF1 gene sequences (including 5′ upstream, 3′ downstream, and intragenic regions) such as those disclosed here are made in vitro and then introduced into the desired plant using standard techniques. Since the efficiency of homologous recombination is known to be dependent on the vectors used, use of dicistronic gene targeting vectors as described by Mountford et al., Proc. Natl. Acad. Sci. USA 91: 4303-4307 (1994); and Vaulont et al., Transgenic Res. 4: 247-255 (1995) are conveniently used to increase the efficiency of selecting for altered OsEMF1 gene expression in transgenic plants. The mutated gene will interact with the target wild-type gene in such a way that homologous recombination and targeted replacement of the wild-type gene will occur in transgenic plant cells, resulting in suppression of OsEMF1 activity.

Alternatively, oligonucleotides composed of a contiguous stretch of RNA and DNA residues in a duplex conformation with double hairpin caps on the ends can be used. The RNA/DNA sequence is designed to align with the sequence of the target OsEMF1 gene and to contain the desired nucleotide change. Introduction of the chimeric oligonucleotide on an extrachromosomal T-DNA plasmid results in efficient and specific OsEMF1 gene conversion directed by chimeric molecules in a small number of transformed plant cells. This method is described in Cole-Strauss et al. Science 273:1386-1389 (1996) and Yoon et al. Proc. Natl. Acad. Sci. USA 93: 2071-2076 (1996).

The endogenous OsEMF1 genes can also be inactivated using recombinant DNA techniques by transforming plant cells with constructs comprising transposons or T-DNA sequences. The OsEMF1 mutants prepared by these methods are identified according to standard techniques.

Other Means for Inhibiting OsEMF1 Activity

OsEMF1 activity may be modulated by eliminating the proteins that are required for EMF1 cell-specific gene expression. Thus, expression of regulatory proteins and/or the sequences that control OsEMF1 gene expression can be modulated using the methods described here.

Another strategy is to inhibit the ability of an OsEMF1 protein to interact with itself or with other proteins. This can be achieved, for instance, using antibodies specific to OsEMF1. In this method cell-specific expression of OsEMF1-specific antobodies is used to inactivate functional domains through antibody:antigen recognition (see, Hupp et al. Cell 83:237-245 (1995)). Alternatively, dominant negative mutants of EMF1 can be prepared. Use of dominant negative mutants to inactivate target genes is described in Mizukami et al. Plant Cell 8:831-845 (1996).

Use of Nucleic Acids of the Invention to Enhance OsEMF1 Gene Expression

Isolated sequences prepared as described herein can also be used to introduce expression of a particular OsEMF1 nucleic acid to enhance or increase endogenous gene expression. For instance, enhanced expression can be used to increase vegetative growth by preventing the plant from making the transition from vegetative to a reproductive state. Where overexpression of a gene is desired, the desired gene from a different species may be used to decrease potential sense suppression effects.

One of skill will recognize that the polypeptides encoded by the genes of the invention, like other proteins, have different domains which perform different functions. Thus, the gene sequences need not be full length, so long as the desired functional domain of the protein is expressed.

Modified protein chains can also be readily designed utilizing various recombinant DNA techniques well known to those skilled in the art and described in detail, below. For example, the chains can vary from the naturally occurring sequence at the primary structure level by amino acid substitutions, additions, deletions, and the like. These modifications can be used in a number of combinations to produce the final modified protein chain.

Preparation of Recombinant Vectors

To use isolated sequences in the above techniques, recombinant DNA vectors suitable for transformation of plant cells are prepared. Techniques for transforming a wide variety of higher plant species are well known and described in the technical and scientific literature. See, for example, Weising et al. Ann. Rev. Genet. 22:421-477 (1988). A DNA sequence coding for the desired polypeptide, for example a cDNA sequence encoding a full length protein, will preferably be combined with transcriptional and translational initiation regulatory sequences which will direct the transcription of the sequence from the gene in the intended tissues of the transformed plant.

For example, for overexpression, a plant promoter fragment may be employed which will direct expression of the gene in all tissues of a regenerated plant. Such promoters are referred to herein as “constitutive” promoters and are active under most environmental conditions and states of development or cell differentiation. Examples of constitutive promoters include the cauliflower mosaic virus (CaMV) 35S transcription initiation region, the 1′- or 2′-promoter derived from T-DNA of Agrobacterium tumafaciens, and other transcription initiation regions from various plant genes known to those of skill. Such genes include for example, ACT11 from Arabidopsis (Huang et al. Plant Mol. Biol. 33:125-139 (1996)), Cat3 from Arabidopsis (GenBank No. U43147, Zhong et al., Mol Gen. Genet. 251:196-203 (1996)), the gene encoding stearoyl-acyl carrier protein desaturase from Brassica napus (Genbank No. X74782, Solocombe et al. Plant Physiol. 104:1167-1176 (1994)), GPc1 from maize (GenBank No. X15596, Martinez et al. J. Mol. Biol 208:551-565 (1989)), and Gpc2 from maize (GenBank No. U45855, Manjunath et al., Plant Mol. Biol. 33:97-112 (1997)). Examples of promoters particularly useful for monocotolydenous plants are described in the literature. Jeon et al., The Plant Journal 22 (6):561-570 (1999); Sentoku et al., Developmental Biology 220:358-364 (2000); Hiei et al., The Plant Journal. 6(2):271-282 (1994); Schaffrath et al., Plant Molecular Biology 43:59-66 (2000).

Alternatively, the plant promoter may direct expression of the OsEMF1 nucleic acid in a specific tissue or may be otherwise under more precise environmental or developmental control. Examples of environmental conditions that may effect transcription by inducible promoters include anaerobic conditions, elevated temperature, or the presence of light. Alternatively, promoter sequences from genes in which expression is controlled by exogenous compounds can be used. For instance, the promoters from glucocorticoid receptor genes can be used (Aoyama and Chau, Plant J 11:605-12(1997)). Such promoters are referred to here as “inducible” or “tissue-specific” promoters. One of skill will recognize that a tissue-specific promoter may drive expression of operably linked sequences in tissues other than the target tissue. Thus, as used herein a tissue-specific promoter is one that drives expression preferentially in the target tissue, but may also lead to some expression in other tissues as well.

Examples of promoters under developmental control include promoters that initiate transcription only (or primarily only) in certain tissues, such as fruit, seeds, or flowers. Promoters that direct expression of nucleic acids in the vegetative shoot apex are particularly useful in the present invention. Examples of suitable tissue specific promoters include the promoter from LEAFY (Weigel et al. Cell 69:843-859 (1992)).

In addition, the promoter sequences from the OsEMF1 genes disclosed here can be used to drive expression of the OsEMF1 polynucleotides of the invention or heterologous sequences. The sequences of the promoters are identified below.

If proper polypeptide expression is desired, a polyadenylation region at the 3′-end of the coding region should be included. The polyadenylation region can be derived from the natural gene, from a variety of other plant genes, or from T-DNA.

The vector comprising the sequences (e.g., promoters or coding regions) from genes of the invention will typically comprise a marker gene which confers a selectable phenotype on plant cells. For example, the marker may encode biocide resistance, particularly antibiotic resistance, such as resistance to kanamycin, G418, bleomycin, hygromycin, or herbicide resistance, such as resistance to chlorosulfuron or Basta.

Production of Transgenic Plants

DNA constructs of the invention may be introduced into the genome of the desired plant host by a variety of conventional techniques. For example, the DNA construct may be introduced directly into the genomic DNA of the plant cell using techniques such as electroporation and microinjection of plant cell protoplasts, or the DNA constructs can be introduced directly to plant tissue using ballistic methods, such as DNA particle bombardment.

Microinjection techniques are known in the art and well described in the scientific and patent literature. The introduction of DNA constructs using polyethylene glycol precipitation is described in Paszkowski et al. Embo J. 3:2717-2722 (1984). Electroporation techniques are described in Fromm et al. Proc. Natl. Acad. Sci. USA 82:5824 (1985). Ballistic transformation techniques are described in Klein et al. Nature 327:70-73 (1987).

Alternatively, the DNA constructs may be combined with suitable T-DNA flanking regions and introduced into a conventional Agrobacterium tumefaciens host vector. The virulence functions of the Agrobacterium tumefaciens host will direct the insertion of the construct and adjacent marker into the plant cell DNA when the cell is infected by the bacteria. Agrobacterium tumefaciens-mediated transformation techniques, including disarming and use of binary vectors, are well described in the scientific literature. See, for example Horsch et al. Science 233:496-498 (1984), and Fraley et al. Proc. Natl. Acad. Sci. USA 80:4803 (1983). In planta transformation procedures can also be used (Bechtold, et al., (1993). Comptes Rendus de l'Academie des Sciences Serie III Sciences de la Vie, 316: 1194-1199; Jeon et al., The Plant Journal 22 (6):561-570 (1999); Sentoku et al., Developmental Biology 220:358-364 (2000); Hiei et al., The Plant Journal. 6(2):271-282 (1994); Schaffrath et al., Plant Molecular Biology 43:59-66 (2000).

Transformed plant cells which are derived by any of the above transformation techniques can be cultured to regenerate a whole plant which possesses the transformed genotype and thus the desired phenotype such as increased seed mass. Such regeneration techniques rely on manipulation of certain phytohormones in a tissue culture growth medium, typically relying on a biocide and/or herbicide marker which has been introduced together with the desired nucleotide sequences. Plant regeneration from cultured protoplasts is described in Evans et al., Protoplasts Isolation and Culture, Handbook of Plant Cell Culture, pp. 124-176, MacMillilan Publishing Company, New York, 1983; and Binding, Regeneration of Plants, Plant Protoplasts, pp. 21-73, CRC Press, Boca Raton, 1985. Regeneration can also be obtained from plant callus, explants, organs, or parts thereof. Such regeneration techniques are described generally in Klee et al. Ann. Rev. of Plant Phys. 38:467-486 (1987).

One of skill will recognize that after the expression cassette is stably incorporated in transgenic plants and confirmed to be operable, it can be introduced into other plants by sexual crossing. Any of a number of standard breeding techniques can be used, depending upon the species to be crossed.

Seed obtained from plants of the present invention can be analyzed according to well known procedures to identify plants with the desired trait. If antisense or other techniques are used to control gene expression, Northern blot analysis can be used to screen for desired plants. In addition, the timing or other characteristics of reproductive development can be detected. Plants can be screened, for instance, for early flowering. Similarly, if OsEMF1 gene expression is enhanced, the plants can be screened for continued vegetative growth. These procedures will depend, part on the particular plant species being used, but will be carried out according to methods well known to those of skill.

### EXAMPLE 1

The following example describes the positional cloning of an EMF1 gene in Arabidopsis.

The EMF1 locus was mapped to the upper arm of chromosome 5, near 20 cM, within an interval of less than 1.5 cM between the molecular markers g6833 and g6830 (Yang et al. Dev. Biol. 169:421-435 (1995)). A Yeast Artificial Chromosome (YAC) clone contig spanning this region was constructed based on published information as well as our own hybridization data. Results obtained from mapping the ends of different YAC clones relative to EMF1 locus showed that the gene resides on the CIC7A7 YAC clone, 2 recombinants away from the right end of yUP18G5 (18G5-R) and 5 recombinants from the CIC9G2 right end (9G2-R). Hence, the 9G2-R and 18G5-R end fragments were used as the closest flanking makers for initiating a chromosome walk from both directions. We screened existing cosmid and lambda genomic libraries, cosmid libraries of the CIC7A7 YAC DNA, and a P1/TAC clone contig spanning the region of CIC7A7, and conducted the further walking to construct a contig consisted of cosmid, lambda, P1 and TAC clone that covers the region from the 18G5-R end to the 9G2-R end. Using polymorphic fragments within these clones to monitor the progress of the chromosome walk towards EMF1 locus, we determined that the EMF1 is located on the cosmid clone CD82.

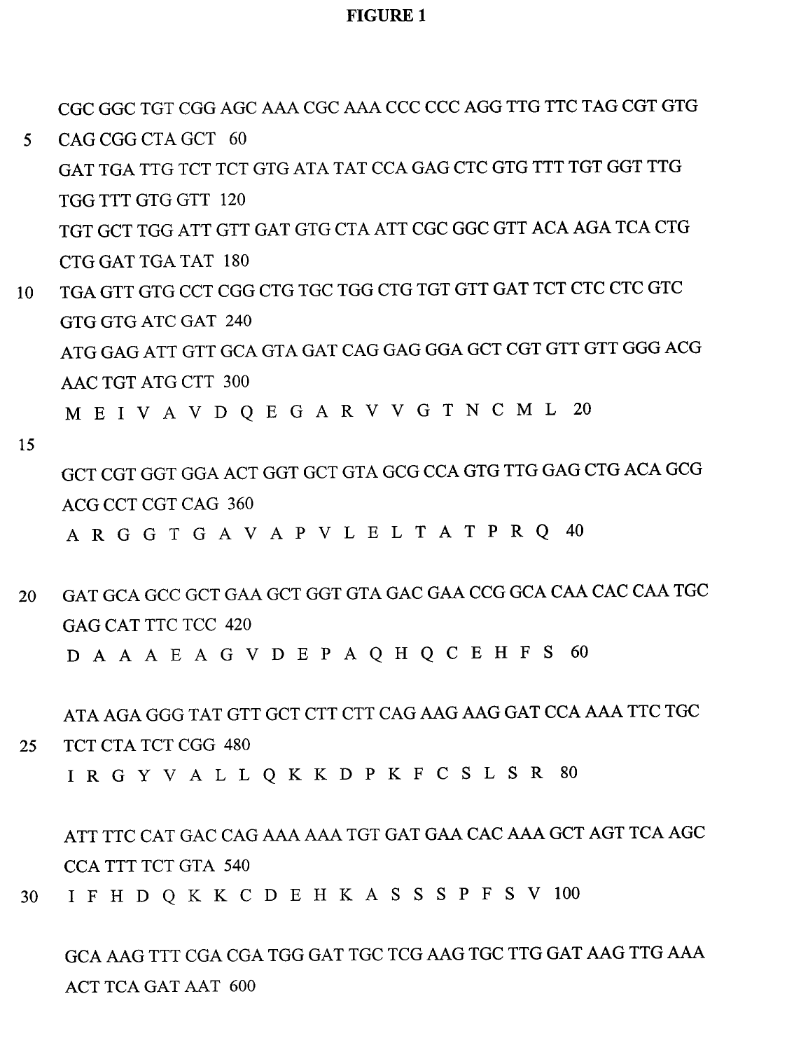

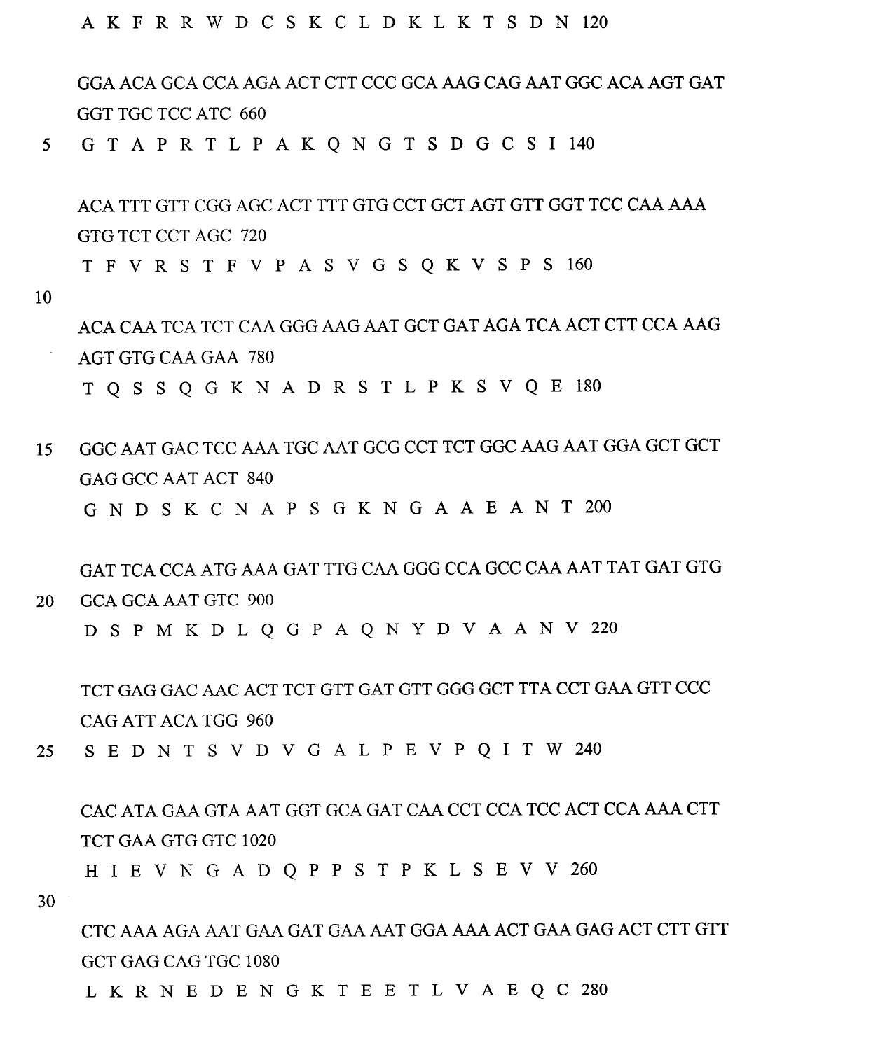

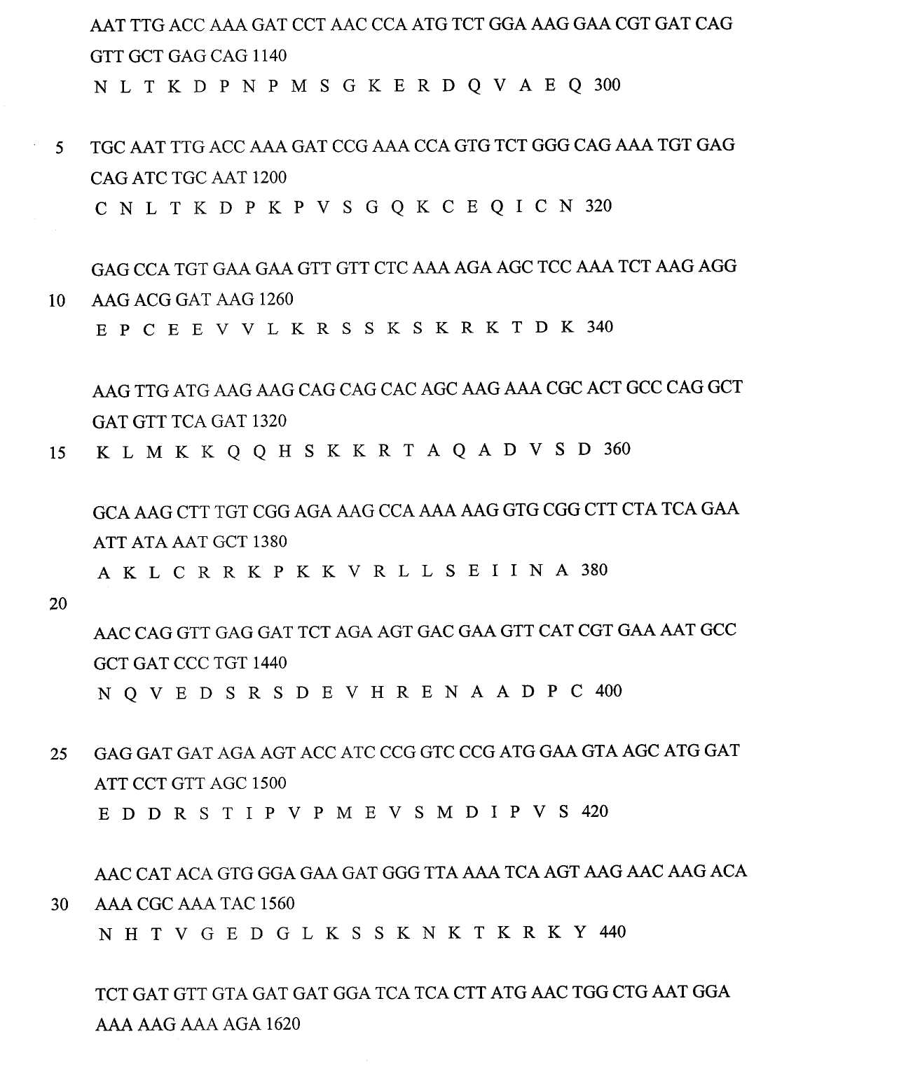

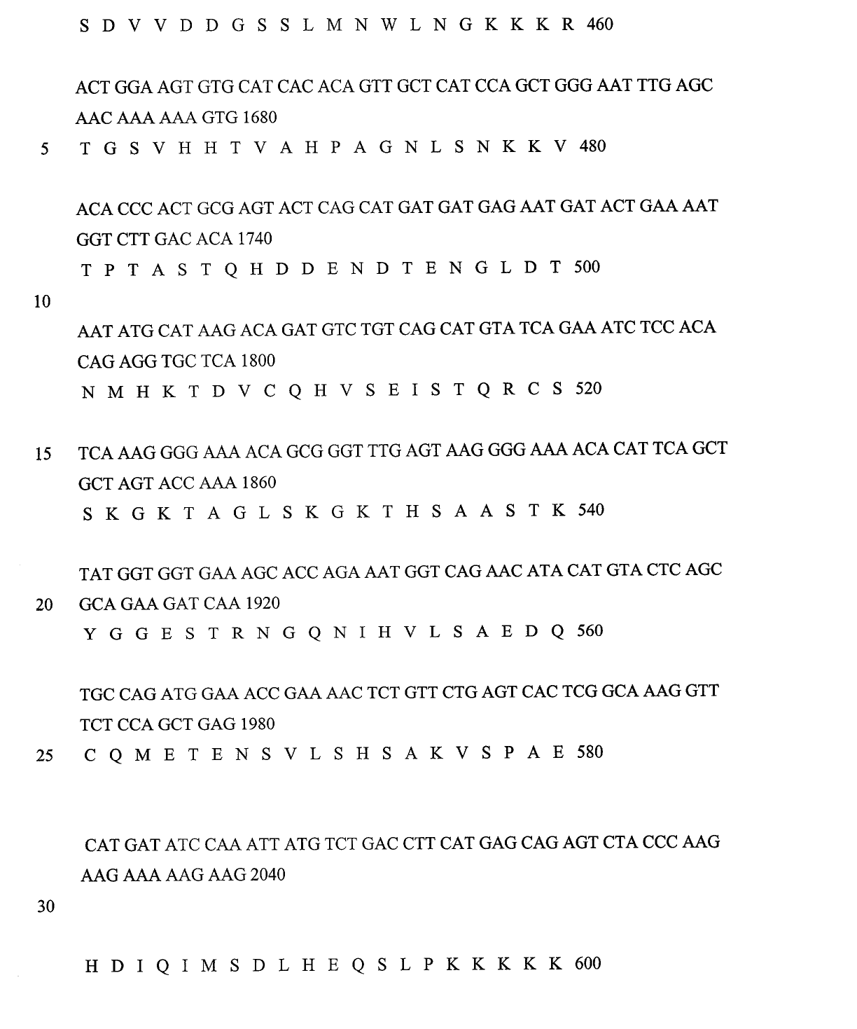

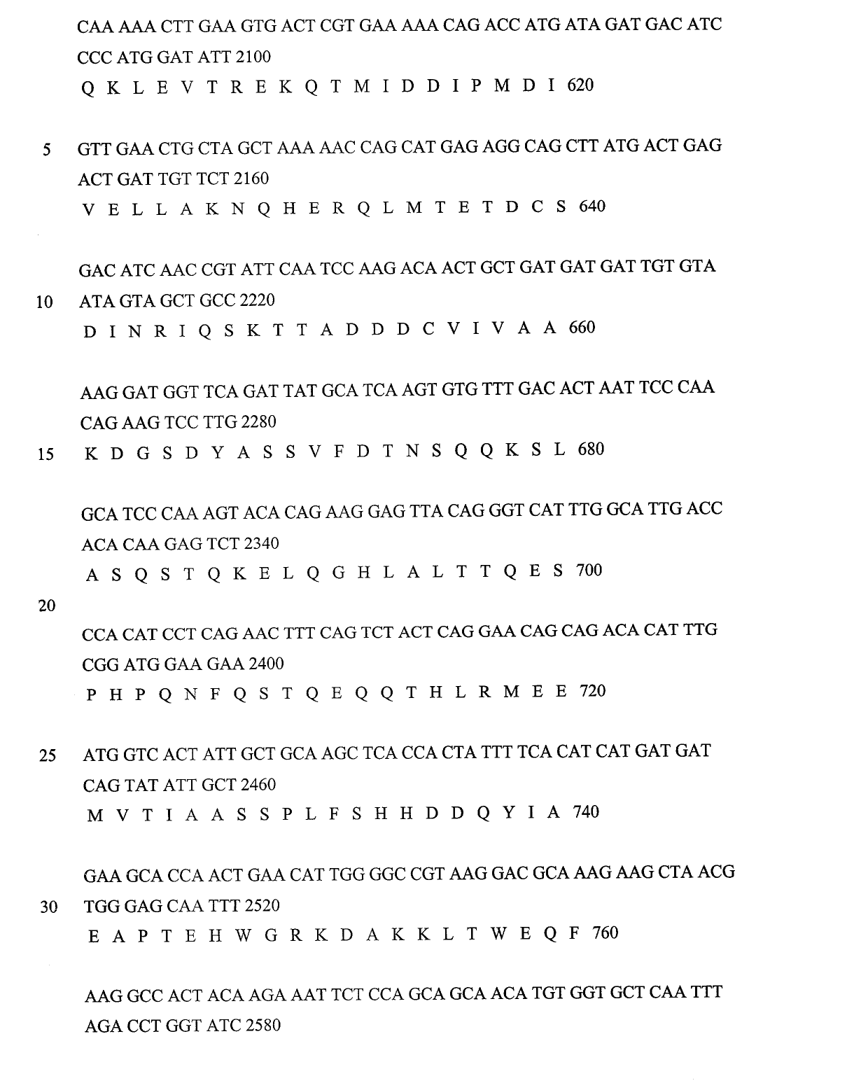

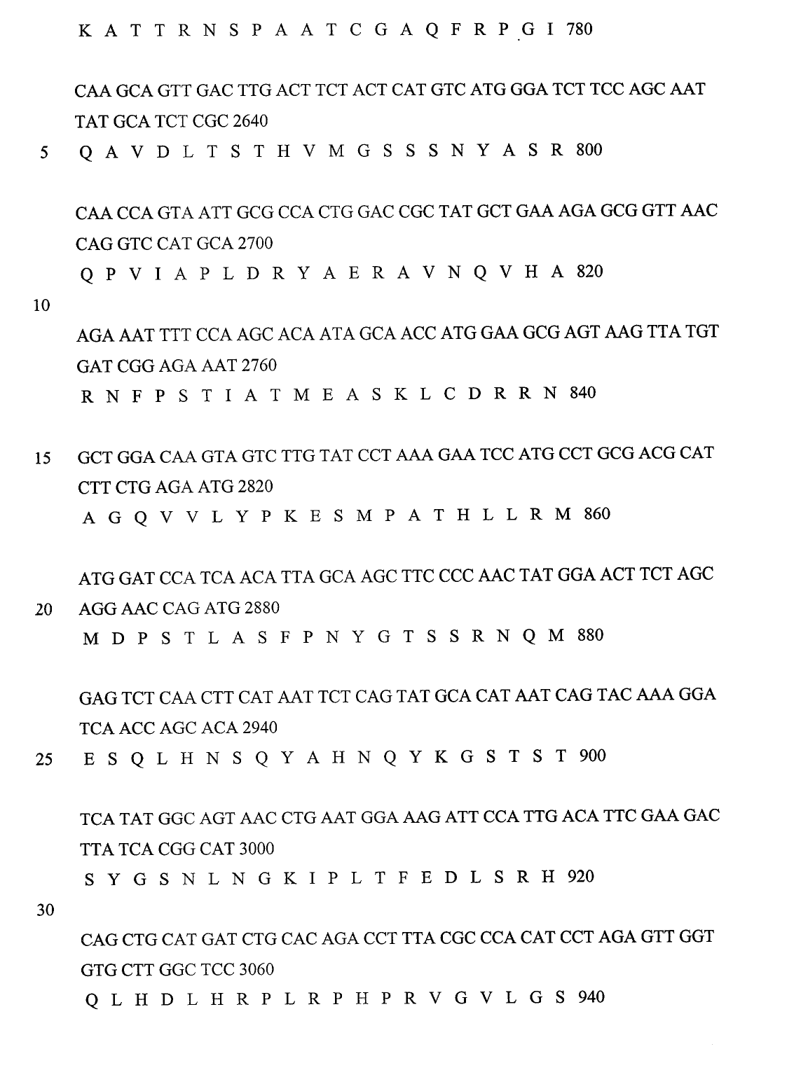

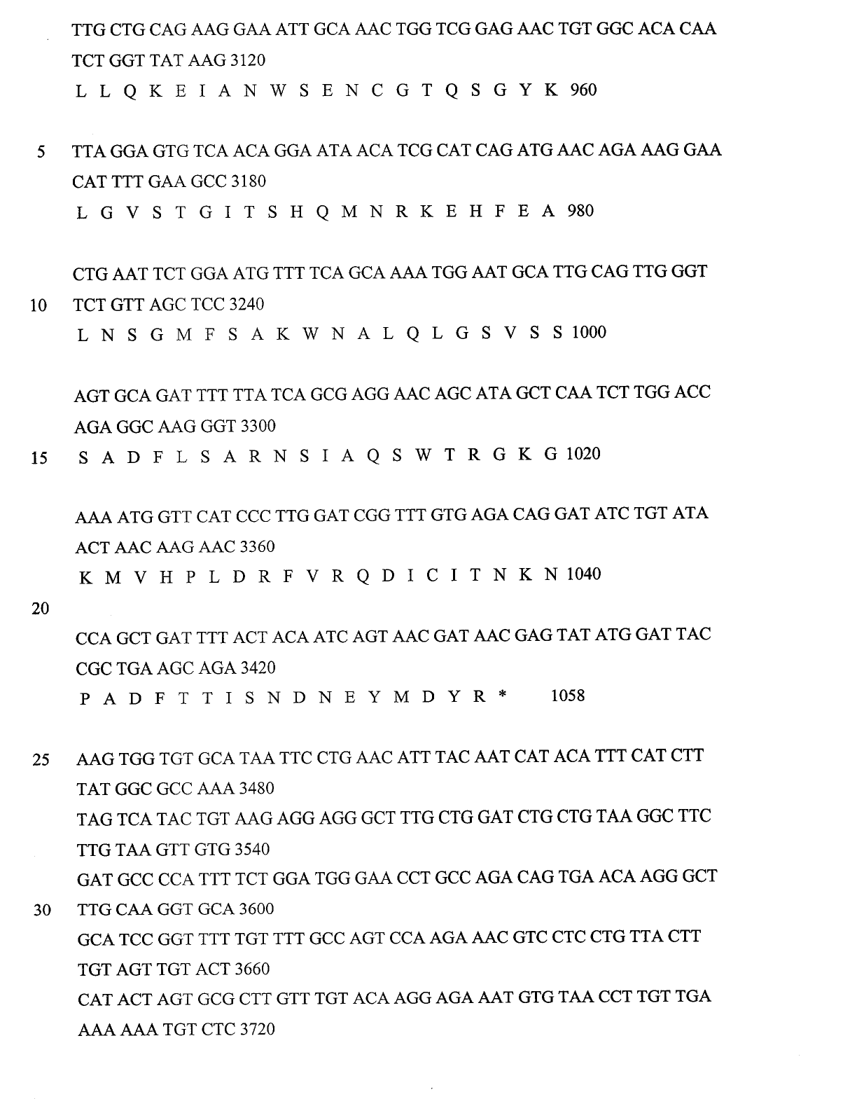

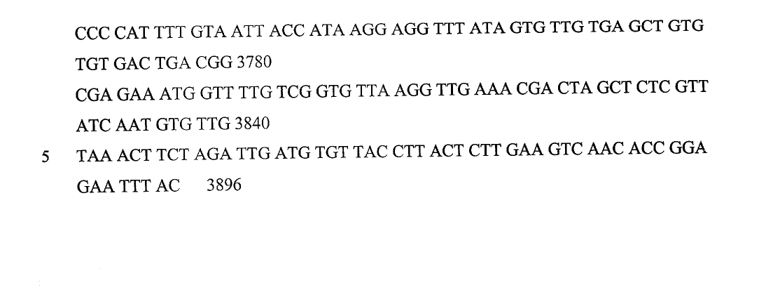

The genomic DNA from CD82 clone was subcloned into pBluescript vector and sequenced (SEQ ID NO: 1). The analysis of CD82 sequence, using DNA Strider program, revealed three ORFs (ORF1, 2, and 3). To define the EMF1 gene among the three ORFs, we mapped the ORF3 using an 1.5 kb BamH1 insert. One recombinant breakpoint was found between EMF1 and the 1.5 kb fragment, ruling out ORF3 as a candidate for EMF1 gene. Based on sequence comparison, we found that ORF2 has homology to gulono-lactone oxiase (23% identities and 41% similarities, gulonolactone oxidase from rattus norvegicus (Nishikimi et al., J. Biol. Chem. 267:21967 (1992) and the Diminuto-like proteins (22% identity and 47% similarity, Wilson et al., Nature 368:32-38 (1994); Takahashi et al., Genes Development 9:97 (1995)). We sequenced the ORF1 gene from plants homozygous for three different emf1 alleles (emf1-1, em1-2 and emf1-3) and identified a frame shift mutation in each of the three alleles (FIG. 1). These frameshift mutations would have resulted in truncated polypeptides. A deletion of 1 base was found at position 2402, 1344, and 941, leading to a truncated protein in emf1-1, 1-2, and 1-3, respectively. The fact that all 3 mutants have a mutation in ORF1 that results in truncated polypetides and that the severity of the mutant phenotypes corresponds with the increased truncation of the polypeptides lead us to conclude that ORF1 is EMF1.

Structural analysis of the EMF1 gene was carried out (SEQ ID NOs:2 and 3). The exon/intron organization of the EMF1 gene was analyzed using NetPlantGene v: 2.0 program (Hebsgaard, et al, Nucleic Acids Research, 24:3439-3452 (1996)). The EMF1 gene consists of 3 exons and 4 introns, and the deduced protein is 931 amino acid long. The sequence comparison using BLAST program against all Arabidopsis GenBank DNA including EST and BAC ends reveals two ESTs with sequence identity to the EMF1 gene. Clone VBVLF01 (from Versailles-VB Arabidopsis thaliana cDNA library, Accession number: Z46543) has 100% identity to EMF1 and clone F2A4T7 (from CD4-14 Arabidopsis thaliana cDNA library, Accession number: N96450) 95%; both are partial cDNA sequences. The comparison using BLAST Program against all non-redundant GenBank CDS translations+PDB+SwissProt+PIR+PRF or dbest (Non-redundant Database of GenBank+EMBL+DDBJ EST Divisions) give no significant similarity to known genes in any organisms, indicating the novelty of the EMF1 gene sequence.

PSORT program (version 6.4 http://psort.nibb.acjp:8800/) was used to predict the subcellular localization of the EMF1 protein. There are two types of nuclear localization signals, both were found in the EMF1 gene. The first type, consisted of three 4 residue patterns composed of basic amino acids (K or R), or three basic amino acids (K or R) and H or P, was found at three positions within the EMF1 protein, i.e., position 231, 347, and 905. The second type of nuclear targeting signal (Robbins et al., Cell, 64:615(1991)), composed of 2 basic residues, 10 residue spacer, and another basic region consisting of at least 3 basic residues out of 5 residues, was found at four different positions, i.e., 78, 106, 217, 905, of the EMF1 protein. Furthermore, the basic residues (K and R) represent 18% of the weight of the protein. This evidence indicates that EMF1 protein is localized in the nucleus.

An ATP/GTP binding site motif A or P-loop ([AG]-X(4)-G-K-[ST]) appears at position 573 in the EMF1 protein. A tyrosine kinase phosphorylation site ([RK-V(2,3)-[DE]-X(2,3)-Y) appears at position 299 in the protein. The LXXLL motif has been proposed to be a signature sequence that facilitates the interaction of different proteins with nuclear receptors (Heery et al. Nature, 387:733 (1997)) was found at position 266. In plants, it has been identified in the RGA protein (a putative transcriptional regulator that represses the gibberellic acid (GA) response, Silverstone et al., Plant Cell, 10: 155 (1998). Another feature of the putative EMF1 protein is the high content in serine residues (S) that represent 10% of the molecular weight, with homopolymeric regions of serine. Taken together the molecular characteristics suggest that EMF1 protein is a novel, transcriptional regulator involved in the flowering signaling pathway.

### EXAMPLE 2

We cloned the rice cDNA, OsEMF1. OsEMF1 is similar to EMF1 in molecular weight, gene structure and functional motifs. Hence, not only Arabidopsis but also a distantly related monocotylcdonous plant, rice, also employs EMF genes in regulating shoot development. Since rice is a major cereal crop, genetic manipulation of OsEMF1 could generate new rice varieties with differing flowering time and seed yield.

The EMF1 gene (GenBank accession number: AF319968) encodes a predicted 121.7 kDA protein (FIG. 2A) with similarity to two Arabidopsis EST clones (GenBank accession number N96450 and Z46543) and to a hypothetical protein from the rice genomic sequencing project (GenBank accession number BAA94774.1, FIG. 2).

To better characterize the rice EMF1 homolog (OsEMF1), we isolated the corresponding cDNA clone by the rapid amplification of cDNA ends (RACE) technique. The OsEMF1 cDNA of 3896 nucleotides (GenBank accession number AF326768) predicts a 1057 amino acid polypeptide (estimated molecular weight, 116.4 kDA) that is 328 amino acids shorter than the predicted protein in BAA94774.1. The organization of introns and exons predicted at the 5′ end in BAA94774.1 was not confirmed by the sequence of the OsEMF1 cDNA (FIG. 2A). The OsEMF1 cDNA is likely to include a complete open reading frame because several stop codons are found in all the three possible reading frames upstream of a first ATG initiating the 1057 amino acid polypeptide. The Arabidopsis and Oryza predicted protein sequences display 37% similarity and 20% identity over their entire length.

Neither EMF1 nor OsEMF1 displays significant homology to proteins of known function from any organism. Nevertheless, several domains could be identified in the predicted EMF1 and OsEMF1 polypeptides (FIG. 2B), including nuclear localization signals (Raikhel, 1992), phosphorylation sites, an ATP/GTP binding motif (P-loop) (Walker et al., 1982), and a LXXLL motif. The LXXLL motif has been demonstrated to mediate the binding of steroid receptor co-activator complexes to a nuclear receptor ((Heery et al. Nature, 387:733 (1997)); Torchia et al., 1997). In plants, it has been identified in the RGA and GAI proteins, both transcriptional regulators in the gibberellic acid (GA) signal transduction pathway (Peng et al., 1997; Silverstone et al., Plant Cell, 10: 155 (1998)). A PSI-BLAST homology search (Altschul et al., 1997) indicates a region of the EMF1 protein between amino acids 901 and 1034 that displays similarity (identities: 23%, positives: 37%) with two members of a nuclear receptor gene family. This gene family comprises one of the most abundant groups of transcriptional regulators in mammals with members involved in various developmental processes (Sluder et al., 1999). Furthermore, the EMF1 protein displays homopolymeric stretches of serine residues, as do the two transcriptonal regulators RGA and GAI, (Silverstone et al., Plant Cell, 10: 155 (1998)). The identification of these motifs indicates that EMF1 and OsEMF1 could represent a new class of regulatory molecules that function as transcriptional regulators during shoot development in higher plants.

According to the present invention, it is possible to create transgenic rice plants that are suppressed in OsEMF1 expression in order to obtain early flowering rice that may result in more crops per year in certain region or may avoid unfavorable growing conditions such as seasonal water shortage, pest attack, low temperature, etc. In addition, according to the present invention, it is desirable to generate rice varieties with more branches for more flowers and higher seed yield.

### EXAMPLE 3

### Antisense EMF1 Plants Display Early Flowering and Shoot Determinacy

To study the function of EMF1, we attempted to decrease EMF1 expression in WT plants. Three constructs containing an EMF1 coding sequence extending 0.6 kb, 2.4 kb, or 3.3 kb from the translation initiation codon in the antisense orientation under the control of the 35S CaMV promoter (35S) were introduced into WT Arabidopsis plants (Bechtold and Pelletier, 1998). The 2,226 T1 transgenic plants carrying the three different antisense constructs displayed a spectrum of emf1-like, early-flowering and WT-like phenotypes. The EMF1-like plants were sterile, while the early-flowering plants were fertile and could grow in the soil. The proportion of the three phenotypic categories observed varied among the constructs. The two longer antisense constructs (2.4 kb and 3.3 kb) gave higher proportions of EMF1-like transgenic plants and lower proportions of early-flowering plants than the shortest construct. The EMF1-like transgenic plants, like EMF1 mutants, lacked rosette leaves and flowered at 14-16 days after sowing. Early-flowering transgenic plants produced 2-8 rosette leaves and flowered at 16-20 days after sowing. In the same growth conditions WT-like plants produced 10-13 rosette leaves and flowered at about 25 days after sowing. The endogenous EMF1 transcript levels of the early-flowering and emf1-like antisense plants were greatly decreased relative to WT-like antisense plants and WT plants.

The above examples are provided to illustrate the invention but not to limit its scope. Other variants of the invention will be readily apparent to one of ordinary skill in the art and are encompassed by the appended claims. All publications, patents, and patent applications cited herein are hereby incorporated by reference.

What is claimed is:

1. An isolated nucleic acid molecule comprising an OsEMF1 polynucleotide sequence, which polynucleotide sequence specifically hybridizes to SEQ ID NO: 1 under stringent conditions.

2. The isolated nucleic acid molecule of claim 1, wherein the OsEMF1 polynucleotide is at least about 100 nucleotides in length.

3. The isolated nucleic acid molecule of claim 1, wherein the OsEMF1 polynucleotide is SEQ ID NO: 1.

4. The isolated nucleic acid molecule of claim 1, further comprising a plant promoter operably linked to the OsEMF1 polynucleotide.

5. The isolated nucleic acid molecule of claim 4, wherein the plant promoter is from a OsEMF1 gene.

6. The isolated nucleic acid of claim 5, wherein the OsEMF1 polynucleotide is linked to the promoter in an antisense orientation.

7. An isolated nucleic acid molecule comprising an OsEMF1 polynucleotide sequence, which polynucleotide sequence encodes an OsEMF1 polypeptide as shown in SEQ ID NO:2.

8. A transgenic plant comprising an expression cassette containing a plant promoter operably linked to a heterologous OsEMF1 polynucleotide of claim 1.

9. The transgenic plant of claim 8, wherein the heterologous OsEMF1 polynucleotide encodes a OsEMF1 polypeptide.

10. The transgenic plant of claim 9, wherein the OsEMF1 polypeptide is as shown in SEQ ID NO:2.

11. The transgenic plant of claim 8, wherein the heterologous OsEMF1 polynucleotide is linked to the promoter in an antisense orientation.

12. The transgenic plant of claim 8, wherein the plant promoter is from an OsEMF1 gene.

13. The transgenic plant of claim 12, wherein the OsEMF1 gene is as shown in SEQ ID NO: 1.

14. A method of modulating reproductive development in a plant, the method comprising introducing into the plant an expression cassette containing a plant promoter operably linked to a heterologous OsEMF1 polynucleotide.

15. The method of claim 14, wherein the heterologous OsEMF1 polynucleotide encodes an OsEMF1 polypeptide.

16. The method of claim 15, wherein the OsEMF1 polypeptide has an amino acid sequence as shown in SEQ ID NO:2.

17. The method of claim 14, wherein the heterologous OsEMF1 polynucleotide is linked to the promoter in an antisense orientation.

18. The method of claim 14, wherein the heterologous OsEMF1 polynucleotide is SEQ ID NO:1.

19. The method of claim 14, wherein the plant promoter is from a OsEMF1 gene.

20. The method of claim 14, wherein the expression cassette is introduced into the plant through a sexual cross.
